(19) United States
(12) Patent Application Publication
(10) Pub. No.: US 2009/0190713 A1
(43) Pub. Date: Jul. 30, 2009

(54) Monitoring Devices, Monitoring Systems, and Methods of Monitoring

(75) Inventor: Kwong Yuen WAI, Aberdeen (HK)

Correspondence Address: RISSMAN HENDRICKS & OLIVERIO, LLP, 100 Cambridge Street, Suite 2101, BOSTON, MA, 02114, US

(73) Assignee: National Electronics & Watch Co. Ltd, Aberdeen (HK)

(21) Appl. No.: 12/021,297

(22) Filed: Jan. 28, 2008

(51) Int. Cl. A61B 5/04; G01C 22/00

(52) U.S. Cl. 377/24.2; 600/509

(57) ABSTRACT

A method of monitoring activities of a user may include receiving information about the user, determining whether the user takes a step, determining the number of steps taken by the user and the length of the steps, and determining at least one of speed of the user, distance traveled by the user, and heart rate of the user.

### TECHNICAL FIELD

The present invention is directed generally to monitoring devices, monitoring systems, and methods of monitoring. More particularly, the present invention is directed to devices and systems for and monitoring of a user's speed and/or distance traversed and/or heart rate, for example, during exercise, sport, or recreational activities.

### BACKGROUND

Conventional monitoring devices are used to monitoring a user's performance during exercise activities. Some conventional monitoring devices include a chest strap heart rate sensor for measuring electrical signals generated on the surface of the user's skin by the beating of the user's heart. The chest strap may include electronics for transforming the heart rate measurements into digital format. The strap may also include a transmitter for transmitting the digital data to a receiver, such as, for example, a receiving watch.

In some conventional monitoring devices, an accelerometer is used to monitor the movement of the user. A signal is generated whenever the user walks or runs. Typically, the accelerometer sensor is mounted in a unit that can be mounted to or incorporated in a shoe.

It may be desirable to provide a chest strap that includes an accelerometer sensor so that the heart rate of and the number of steps taken by the user can be combined and sent to a receiver at the same time. Such an arrangement may eliminate the need for a shoe-mounted unit for calculating speed and distance of the user. It may be desirable to provide a monitoring device that determines whether a user is walking or running and determines the speed of and distance traversed by the user based thereon.

### SUMMARY

In accordance with various aspects of the disclosure, a monitoring device may comprise an input device configured to receive inputs of information about a user, at least one first sensor configured to sense vertical acceleration of the user, at least one second sensor configured to sense heart rate of the user, and a controller configured to receive the information about the user. The controller is configured to determine whether the user takes a step, the number of steps taken by the user and the length of the steps, and at least one of speed of the user, distance traveled by the user, and heart rate of the user.

In some aspects of the disclosure, a method of monitoring activities of a user may comprise receiving information about the user, determining whether the user takes a step, determining the number of steps taken by the user and the length of the steps, and determining at least one of speed of the user, distance traveled by the user, and heart rate of the user.

### BRIEF DESCRIPTION OF THE DRAWINGS

FIG. 1 illustrates a partial perspective view and a partial top plan view of an exemplary monitoring system in accordance with various aspects of the disclosure.

FIG. 2 is a block diagram of the exemplary monitoring system of FIG. 1 in accordance with various aspects of the disclosure.

FIG. 3 illustrates exemplary heart rate pulses in accordance with various aspects of the disclosure.

FIG. 4 is a flow chart illustrating an exemplary method of monitoring in accordance with various aspects of the disclosure

FIG. 5 illustrates a partial perspective view and a partial top plan view of an exemplary monitoring system in accordance with various aspects of the disclosure.

FIG. 6 is a block diagram of the exemplary monitoring system of FIG. 5 in accordance with various aspects of the disclosure.

FIG. 7 is a block diagram of an exemplary accelerometer sensor in accordance with various aspects of the disclosure.

FIGS. 8A-8E illustrate exemplary accelerometer readings and associated processed signals in accordance with various aspects of the disclosure.

### DETAILED DESCRIPTION

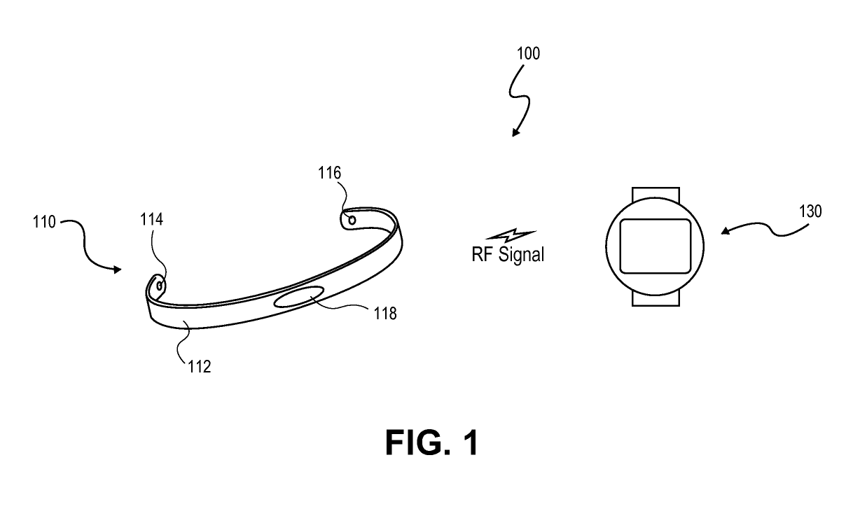

An exemplary embodiment of a monitoring system in accordance with various aspects of the disclosure is illustrated in FIG. 1. Although various aspects of the disclosure are directed to a timepiece, it should be appreciated that the various aspects may also pertain to other electronic devices such as, for example, wrist-worn electronic devices, handheld devices, and portable devices. These devices may include audio players, video players, monitors, or the like.

FIG. 1 shows a partial perspective view and a partial top plan view of a monitoring system 100. The monitoring system may include a monitoring device 110 and an output member 130. The monitoring device 110 may comprise, for example, a strap 112 configured to be attached to a user's chest. The monitoring device 110 may include a first electrode 114 and a second electrode 116 that are arranged on the strap 112 so as to contact the user's skin at the chest. The electrodes may thus pick up electrical signals from the heartbeat. The monitoring device 110 may include an accelerometer 118. The accelerometer 118 may detect the motion of a user wearing the strap 112.

The output member 130 may be separate from the monitoring member 110. The monitoring member 110 may be configured to electrically communicate with the output member 130, for example, via wireless communication. According to various aspects, the monitoring member 110 and the output member 130 may be configured to communicate with one another via radio frequency (RF) signals, Bluetooth signals, or the like. In some aspects, the monitoring member 110 may be electrically coupled with the output member 130 via an electrical wire (not shown), and communication signals may be sent from the monitoring member 110 to the output member 130, and vice versa, via the electrical wire.

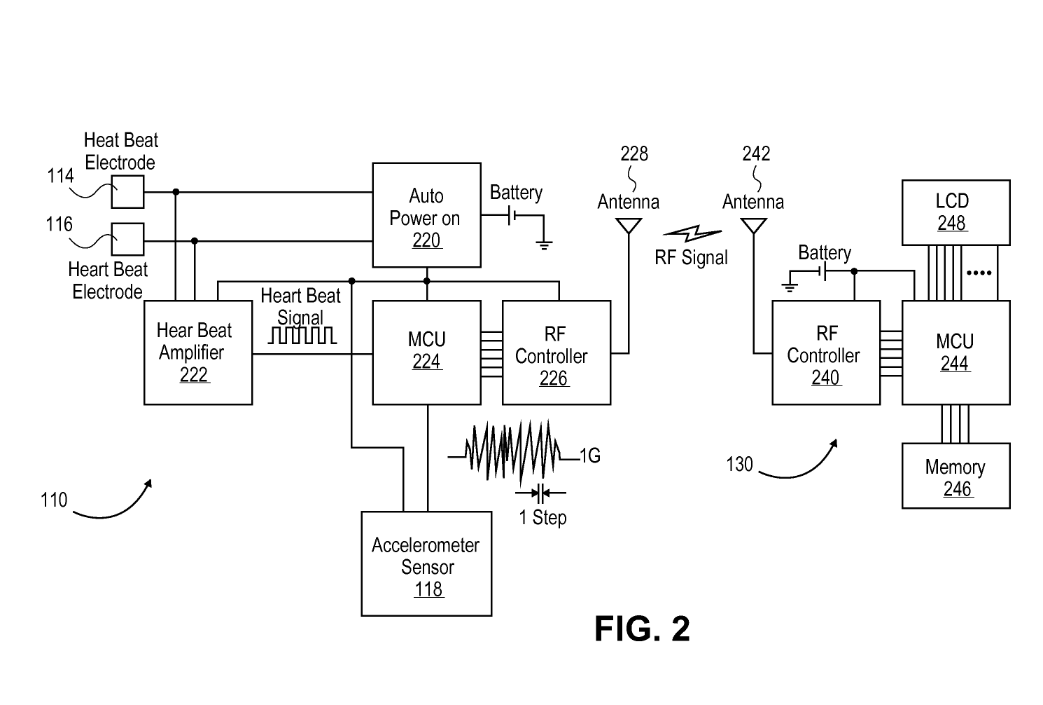

Referring now to FIG. 2, a block diagram of the monitoring system 100 is shown. The first and second electrodes 114, 116, when in contact with a user's skin, may enable an auto power on circuit 220 and provide power to the circuitry of the monitoring device 110. The electrodes 114, 116 may be configured to detect the weak electrical signals generated by a user's heartbeat.

The electrodes 114, 116 may be electrically coupled with an amplifier 222 so that outputs of the electrodes 114, 116 may be directed to the amplifier 222. The amplifier 222 may be electrically coupled with a microcontroller (MCU) 224 so that outputs of the amplifier 222 may be directed to the microcontroller 224. The accelerometer 118 may also be electrically coupled with the microcontroller 224 so that outputs of the accelerometer 118 are directed to the microcontroller 224.

The microcontroller 224 may be instructed to calculate a user's heart rate and/or to calculate steps taken by the user. The microcontroller 224 may be electrically coupled with a transmitter/receiver 226, which may be configured to transmit the calculated heart rate and/or motion information and/or to receive information via an antenna 228. In some aspects, the transmitter/receiver 226 may comprise an RF controller.

As shown in FIG. 2, the output member 130 may include a transmitter/receiver 240 configured to receive the calculated heart rate and/or acceleration information from the transmitter/receiver 226 and/or to transmit information via an antenna 242. The output member 130 may also include a microcontroller (MCU) 244 electrically coupled with the transmitter/receiver 240. The microcontroller 244 may be configured to determine characteristics of a user's activities, such as, for example, the user's speed and distance traversed. According to some aspects, the output member 130 may include a memory 246 configured to store heart rate and/or user motion information. The output member 130 may also include a display 248, for example, a liquid crystal display (LCD), configured to display a user's heart rate information and/or information related to a user's activities.

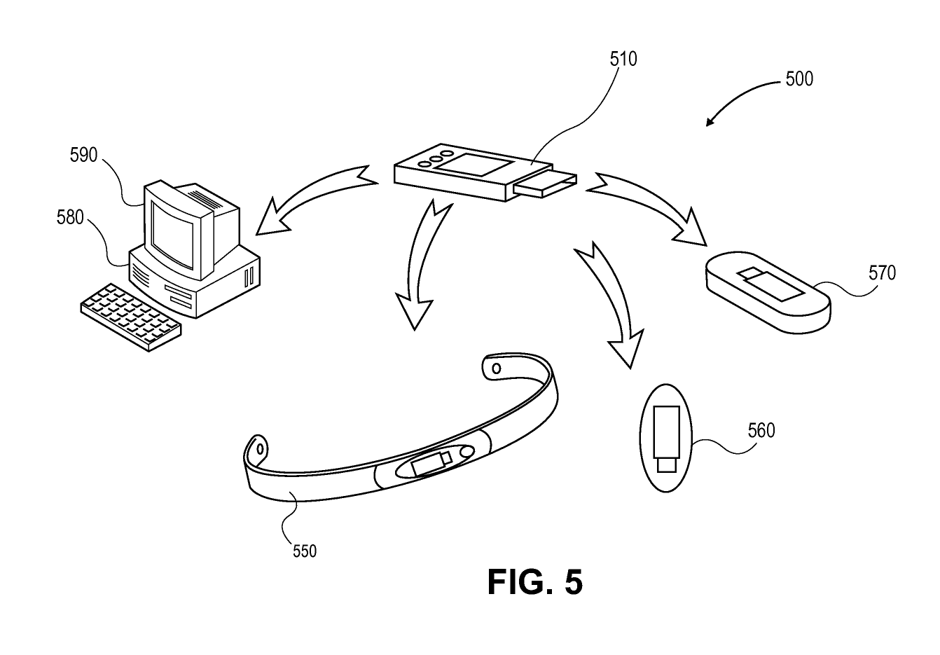

According to various aspects of the disclosure, FIG. 5 shows a partial perspective view and a partial front plan view of a monitoring system 500. The monitoring system 500 may include a monitoring device 510, such as, for example, a USB device. The system 500 may further include one or more housings configured to receive the monitoring device 510. The housings may be structured and arranged so as to be associated with various types of apparel and/or footware. For example, according to various aspects, the one or more housings may include a chest strap 550, a belt clip 560, and/or a footpod 570. The chest strap 550 may be secured about the chest of a user by any known attachment means, the belt clip 560 may include any known means for coupling to a belt, a belt loop, a waistband, or the like, and the footpod 570 may be received by a complementary opening in a shoe or otherwise coupled to a shoe by any know means.

The monitoring system 500 may also include a processing device 580, such as, for example, a personal computer. According to various aspects, the processing device may comprise a notebook computer, a personal digital assistant, a pocket personal computer, or the like. The processing device 580 may be configured to receive data from the monitoring device 510 and to store, process, and/or output the data or any information derivable therefrom. A display 590 may be integral with, electrically coupled with, and/or separate from the processing device 580.

The chest strap 550 may include a first electrode 554 and a second electrode 556 that are arranged on the strap 550 so as to contact the user's skin at the chest. The electrodes 554, 556 may thus pick up electrical signals from the heartbeat.

The monitoring device 510 may include an accelerometer 518. The accelerometer 518 may be configured to detect and/or monitor movement of a user with whom the monitoring device 510 is associated. The monitoring member 510 may be configured to electrically communicate with the processing device 580 or a device such as output member 130, for example, via a USB connection or other wired connection, or via wireless communication. The wireless communication may comprise radio frequency (RF) signals, Bluetooth signals, or the like. In some aspects, the monitoring device 510 may be electrically coupled with the processing device 580 via an electrical connection or via an electrical wire (not shown), and communication signals may be sent from the monitoring device 510 to the processing device 580, and vice versa, via the electrical wire or electrical connection.

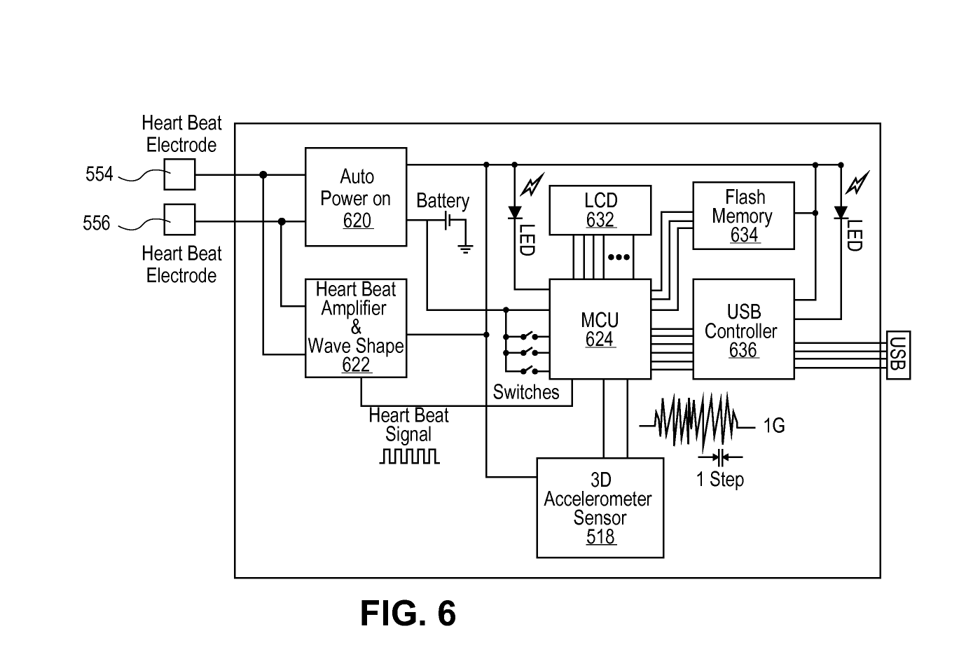

Referring now to FIG. 6, a block diagram of the monitoring system 500 is shown. The first and second electrodes 554, 556 of the strap 552, when in contact with a user's skin, may enable an auto power on circuit 620 and provide power to the circuitry of the monitoring device 510. The electrodes 554, 556 may be configured to detect the weak electrical signals generated by a user's heartbeat.

The electrodes 554, 556 may be electrically coupled with an amplifier 622 so that outputs of the electrodes 554, 556 may be directed to the amplifier 622. The amplifier 622 may be electrically coupled with a microcontroller (MCU) 624 so that outputs of the amplifier 622, for example, heartbeat signals, may be directed to the microcontroller 624. The accelerometer 518 may also be electrically coupled with the microcontroller 624 so that outputs of the accelerometer 518, for example, acceleration data and/or step/stride signals, are directed to the microcontroller 624.

The microcontroller 624 may be instructed to calculate a user's heart rate and/or to calculate steps taken by the user. The microcontroller 624 may be electrically coupled with a battery (not numbered), a liquid crystal display (LCD) 632, memory 634, and a USB controller. The LCD 632 may be configured to display information to a user. Memory 246 may be configured to store heart rate and/or user motion information, for example, via flash memory or the like.

The USB controller 636 may be configured to transmit the calculated heart rate and/or motion information and/or to receive information via, for example, a USB electrical connector 638. In some aspects, the monitoring device may include a transmitter/receiver (not shown) configured to communicate wirelessly with the processing device 580.

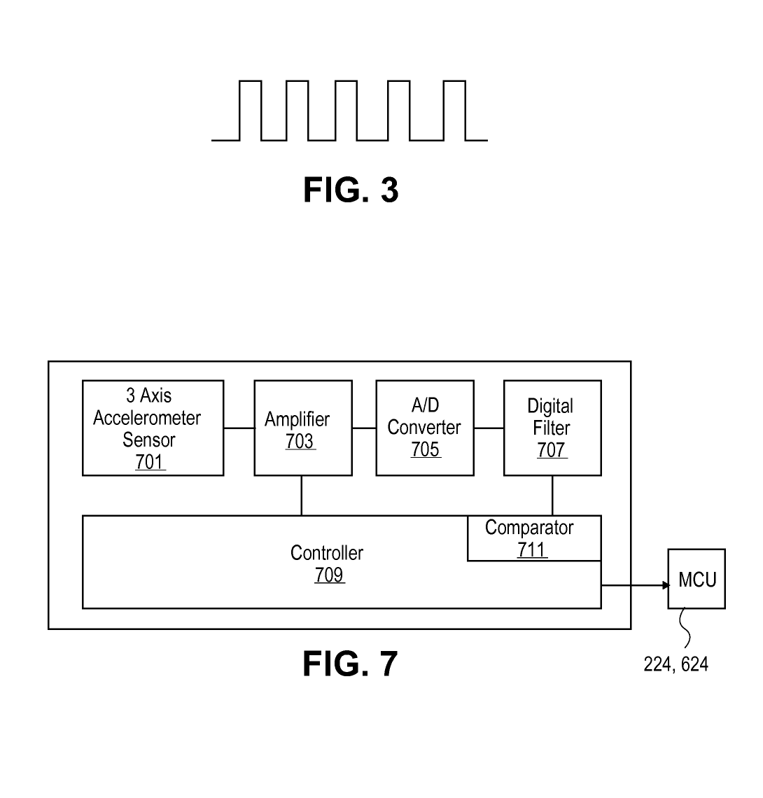

Referring now to FIG. 3, an exemplary waveform of a user's heartbeat is shown. Electrical signals generated by the user's heartbeat may be detected by the electrodes 114, 116, 554, 556, amplified by the amplifier 222, 522, and processed by the microcontroller 224, 624 to represent the user's pulse. The period between the beginnings of adjacent pulses is the period of the heartbeat.

FIG. 7 illustrates an exemplary accelerometer block diagram. As shown, the accelerometer 118, 518 may include, for example, a three-axis accelerometer sensor 701 configured to sense movement of a user and output acceleration data, which is then amplified via an amplifier 703 and directed to an analog-to-digital (A/D) converter 705. Data output from the A/D converter 705 is then passed through a digital filter 707 before being directed to a controller 709. The controller 709 may include a comparator 711 configured to check the outputs of the digital filter 707. The controller 709 may be configured to control the order of the amplification and/or filtering so as to improve the signal-to-noise ratio of the acceleration data. Step and/or acceleration data may be directed from the accelerometer 118, 518 to the microcontroller 224, 624, for example, by the controller 709.

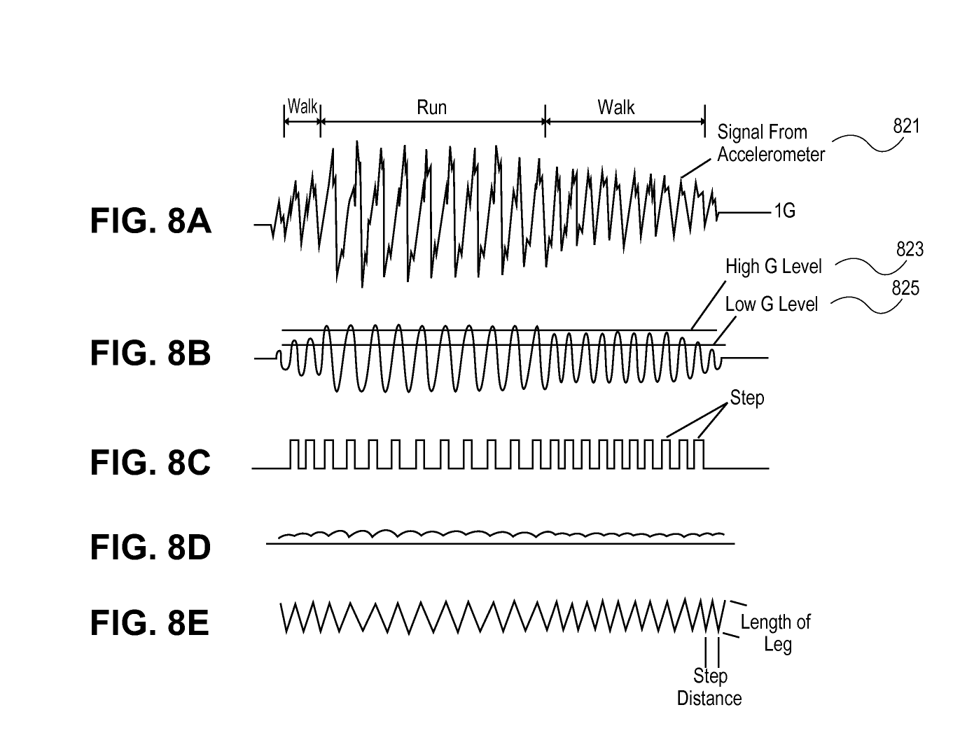

Referring now to FIGS. 8A-8E, an exemplary waveform of the accelerometer during walking and running is illustrated. FIG. 8A shows a signal 821 from the accelerometer sensor 701 after amplification by the amplifier 703. FIG. 8B shows the signal 821 after noise is filtered from the signal by the digital filter 707. As previously mentioned, the controller 709 may control the order of filtering by the filter 707 in order to improve the signal-to-noise ratio. The controller 709 can set two threshold levels 823, 825, for example, a high G level and a low G level. These levels are set to allow the comparator 711 to determine when a user equipped with the monitoring device 110, 310 is walking or running. For example, when the signal 821 meets or exceeds the high G level 823, the comparator 711 determines that the user is running, and when the signal exceeds the low G level 825 but does not exceed the high G level 823, the comparator 711 determines that the user is walking.

FIG. 8C illustrates a digitized form of the signal 821 so that the number of steps/strides can be counted and/or recorded, for example, in memory 634. FIG. 8D illustrates the signal 821 processed to reflect the minimum acceleration below normal 1 G; that is, 1 G-AZMIN, where AZMIN is the minimum acceleration recorded by the accelerometer 118, 518. The MCU 224, 624 can monitor the value of acceleration recorded by the accelerometer sensor 118, 518. FIG. 8E illustrates a signal representative of the step distance, which can be calculated from the length of the user's leg and the acceleration data sensed and/or recorded.

The length of the user's leg may be calculated based on the user's body height, which information can be input into the monitoring system 100, 500. For example, according to various aspects, the leg length can be determined based on a fraction or percentage of the user's height. In some aspects, the leg length may be equal to 45% or 0.45 of the user's body height. Other percentages can be determined and the system modified to reflect the typical or average relationship between leg length and body height. In some aspects, the relationship between leg length and body height may differ between male and female users. Accordingly, the system may be programmed to prompt a user for input of his/her sex and then the appropriate relationship can be used to determine the leg length based on the user's inputted body height. The system 100, 500 may also prompt a user to enter various other personal data such as weight, age, etc.

Once the user's leg length is determined, step length can be determined according to the following formula:

SL=2*A*SQR(LH)*SQR(1 G−AZMIN) (1)

where SL=step length
LH=leg length
(1 G−AZIMIN)=minimum acceleration less than 1 G
A=constant
   The constant A can be determined based on evaluation of a sampling study or the like.

The distance traversed by the user can then be determined as follows:

DISTANCE=SL*N_STEP (2)

where SL=step length computed according to equation (1)
N_STEP=the number of steps recorded

After computing the distance, the user's speed, or velocity, can be computed by dividing the DISTANCE calculated in equation (2) by the time period over which the monitoring device 110, 510 is sensing the user's movement. The time may be tracked and/or recorded, for example, by the microcontroller 124, 624.

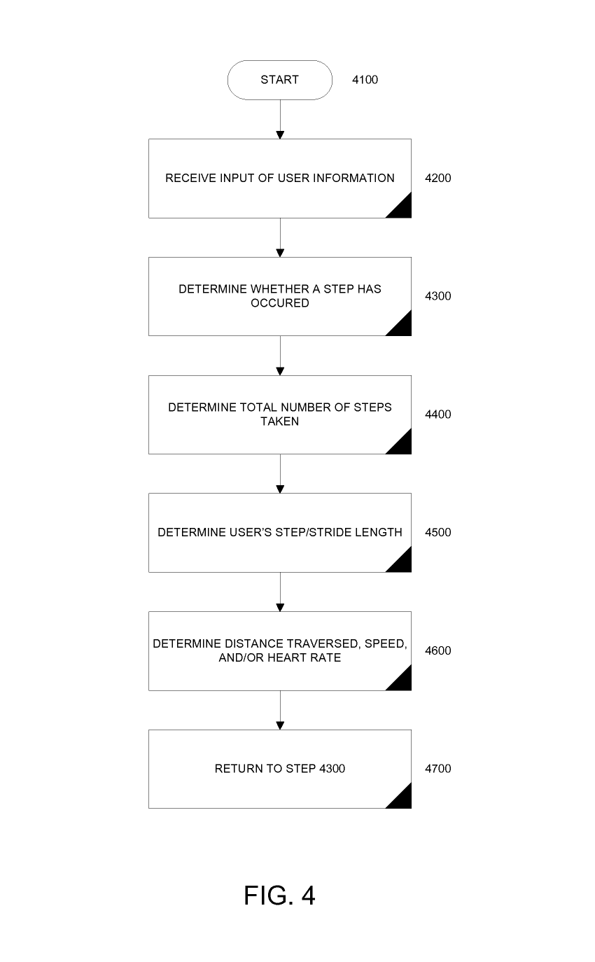

Referring now to FIG. 4, a flowchart illustrates some of the basic steps associated with an exemplary method of monitoring a user's heart rate and/or the speed and distance traversed by the user. The process begins at step 4100. The process may be commenced, for example, when the monitoring device 110, 510 is associated with a user and activated, either by a user input or by the auto power on circuit 220, 620, which may be enabled via contact between the electrodes 114, 116, 554, 556 and the user's skin. Control then continues to step 4200.

In step 4200, the system 100, 500 receives inputted information about the user such as, for example, user height, user sex, user weight, user age, or the like. This information may be input via an input interface (unnumbered) associated with the monitoring device 110, 510, output member 130, and/or the processing device 580. Next, in step 4300, the controller 124, 624 determines whether a step has occurred. This determination may include a sub-process for analyzing the measurements of the accelerometer 118, 518 and determining when the vertical acceleration moves beyond a threshold, for example, from below a low G level 825 to above the low G level 825 and/or above the high G level 823. Control then proceeds to step 4400, where the total number of steps taken is determined and/or recorded, as reflected in FIG. 8C. Control continues to step 4500.

In step 4500, the system 100, 500 determines the user's step/stride length based on the amplitude of the minimal vertical acceleration sensed by the accelerometer 118, 518 and the user's leg length in accordance with, for example, equation (1). As described above, the leg length may be determined based on the user's height, which may be input by prompting the user to enter his or her height. The stride length is substantially proportional to the user's body height, and thus the user's leg length, but varies depending on whether the user is running or walking. Control proceeds to step 4600, where the system 100, 500 determines the distance traversed by the user, the user's speed, or velocity, and/or the user's heart rate. Of course, the user's heart rate can be determined directly from the heart rate sensors and without steps 4400 and 4500. Thus, one skilled in the art would recognize that such variations of this exemplary process are encompassed by this disclosure. Moreover, the above-mentioned determinations of distance traversed, speed, etc. can be additionally or alternatively computed in a continuous manner such that the speed is an average speed of the user for a period of time or an instantaneous speed of the user. Additional computations such as total calories burned, calories per unit time burned, etc. are well known and encompassed by this disclosure. Control then proceeds to step 4700, where the process returns to step 4300 and continues until the user stops taking steps and/or the system 100, 500 is powered off.

It will be apparent to those skilled in the art that various modifications and variations can be made in the monitoring devices, monitoring systems, and methods of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A monitoring device, comprising:
an input device configured to receive inputs of information about a user;
at least one first sensor configured to sense vertical acceleration of the user;
at least one second sensor configured to sense heart rate of the user; and
a controller configured to receive said information about the user, determine whether the user takes a step, determine the number of steps taken by the user and the length of the steps, and determine at least one of speed of the user, distance traveled by the user, and heart rate of the user.

2. The device of claim 1, further comprising an output member configured to display at least one of the speed of the user, the distance traveled by the user, and heart rate of the user.

3. The device of claim 1, wherein the controller is configured to determine whether the user takes a step by determining whether vertical acceleration of the user exceeds a threshold value.

4. The device of claim 3, wherein the controller is further configured to determine the step length based on an amplitude of the vertical acceleration and leg length of the user.

5. The device of claim 4, wherein said user information includes the user's height, and the controller is further configured to determine the leg length of the user from a height of the user.

6. The device of claim 1, wherein the device comprises:
a first member including said at least one first sensor and said at least one second sensor, said first member being configured to be coupled to the user's body;
a second member including said display.

7. The device of claim 6, wherein the second member is configured to be coupled to the user, the first member being configured to wirelessly communicate with the second member.

8. The device of claim 6, further comprising a plurality of housings, each of said plurality of housings being configured to receive the first member.

9. The device of claim 8, wherein each of said plurality of housings is configured to couple the first member to the user's body.

10. The device of claim 8, wherein the first member comprises a USB device.

11. The device of claim 10, wherein the second member is configured to be coupled to the user, the first and second members being configured to wirelessly communicate with one another.

12. The device of claim 10, wherein the second member comprises one of a desktop computer, a notebook computer, and a tablet computer.

13. The device of claim 12, wherein the first and second members are configured to communicate wirelessly with one another.

14. The device of claim 12, wherein the second member comprises a USB port configured to receive the first member.

15. The device of claim 8, wherein said plurality of housings includes at least two of the following: a chest strap, a belt clip, and a footpod.

16. A method of monitoring activities of a user, the method comprising:
receiving information about the user;
determining whether the user takes a step;
determining the number of steps taken by the user and the length of the steps; and
determining at least one of speed of the user, distance traveled by the user, and heart rate of the user.

17. The method of claim 16, further comprising outputting at least one of the speed of the user, the distance traveled by the user, and heart rate of the user.

18. The method of claim 16, wherein the step of determining whether the user takes a step includes determining whether vertical acceleration of the user exceeds a threshold value.

19. The method of claim 18, further comprising determining the step length based on an amplitude of the vertical acceleration and leg length of the user.

20. The method of claim 19, wherein said user information includes the user's height, the method further comprising determining the leg length of the user from a height of the user.
